(19) United States
(12) Patent Application Publication
(10) Pub. No.: US 2006/0210488 A1
(43) Pub. Date: Sep. 21, 2006

(54) Teeth whitening candy with tartar removal and breath freshening properties

(76) Inventor: Henryk P. Jakubowski, Forest Hills, NY (US)

Correspondence Address: Henryk P. Jakubowski, 65-10 108st Apt 5H, Forest Hills, NY, 11375-1827, US

(21) Appl. No.: 11/084,468

(22) Filed: Mar. 19, 2005

(51) Int. Cl. A61K 9/68

(52) U.S. Cl. 424/48

(57) ABSTRACT

This invention discloses a teeth whitening candy intended to fill the gap between daily brushing and the need to augment dental hygiene between visits with dental professionals. The candy will enhance the removal of the tartar and plaque, which accumulates after meals. In addition to whitening teeth, the candy will help to dissolve tartar, calculus and stains from the various surfaces of teeth. Although the candy is intended as stand alone product, it will be also useful in maintaining/prolonging the effects of professional whitening procedures.

### CROSS-REFERENCE TO RELATED APPLICATIONS

| | |
|---|---|
| 6843981 Jan. 18, 2005 Ishibashi | 424/53 |
| 6361320 Mar. 26, 2002 Yarborough | 433/215 |
| 6610276 Aug. 26, 2003 Melman | 424/57 |
| 6121213 Sep. 19, 2000 Vergara | 510/115 |
| 4837008 Jun. 6, 1989 Rudy | 424/52 |
| 4925655 May 15, Smigel | 424/52 |
| 4405599 Sep. 20, 1983 Smigel | 424/53 |
| 4603045 Jul. 29, 1986 Smigel | 424/52 |
| 5843471 Dec. 1, 1998 Chaikin | 424/440 |
| 6013274 Jan. 11, 2000 Chaikin | 424/440 |
| 6090402 Jul. 18, 2000 Chaikin | 424/440 |
| 3590120 Jun. 29, 1971 Muhler | 424/48 |
| 5980641 Nov. 9, 1999 Jakubowski | 134/1 |
| 4170633 Oct. 9, 1979 Wagenknecht | 424/48, 424/49 |
| 6696043 Feb. 24, 2004 Orlowski | 424/48 |
| 6846500 Jan. 25, 2005 Luo | 426/3 |
| 6471945 Oct. 29, 2002 Luo | 424/48 |
| 6485739 Nov. 26, 2002 Luo | 424/440 |
| 6696044 Feb. 24, 2004 Luo | 424/48 |
| 5824291 Oct. 20, 1998 Howard | 424/48 |
| 5972374 Oct. 26, 1999 Theisen | 424/440 |
| 6416744 Jul. 9, 2002 Robinson | 424/48 |

### STATEMENT OF FEDERALL SPONSORED RESEARCH OR DEVELOPMENT

Not applicable

### BACKGROUND OF INVENTION

A recent trend in teeth whitening procedures in dental offices, in home use kits, in various brands of tooth pastes, and/or chewing gum, utilizes one common procedure: introduces bleaching agents to the surfaces of the teeth in order to whiten teeth. Hydrogen peroxide is one of the most common bleaching agent employed in the process.

### FIELD OF INVENTION

This invention relates to field of teeth whitening, and utilizes sugar free candy comprising of citric acid, calcium peroxide, sodium bicarbonate, titanium dioxide, and tannic acid with other flavors and artificial sweeteners as a means to saliva stimulation. Calcium peroxide combined with citric acid dissolved by saliva will produce hydrogen peroxide as a bleaching agent. The acidic nature of the outer part of candy will affect tartar and calculus present on the surfaces of the teeth. This portion of the candy will dissolve tartar and calculus, and will aid in their removal from the surfaces of the teeth. The alkaline inner portion of the candy will contain titanium dioxide and sodium bicarbonate dissolved by saliva and will be optionally combined with any organic gum/thickening agent. This will then coat surfaces of the teeth and mask un-removed stains to produce whiter teeth.

### DETAILED DESCRIPTION OF PRIOR ART

In the prior art there are several methods of bleaching and whitening teeth. The most effective treatment for whitening teeth is professional bleaching, preformed in a dental office. While effective, there are several drawbacks to the procedure: professional whitening requires making a dental appointment, use of harsh chemicals to achieve desired effects quickly. Typically office systems comprise of bleaching agents in concentrations upwards of 25% of hydrogen peroxide, or carbamide peroxide. Titanium dioxide in concentration of 10% is often added to initiate photocatalitic reactions: “use of titanium dioxide in initiating photocatalitic action with light irradiation” see Pat. No. 6,843,981 by Ishibashi issued on Jan. 18, 2005.

Bleaching agents are usually delivered in a gel form, often additionally activated by laser light: see Pat. No. 6,843,981 by Ishibashi issued on Jan. 18, 2005. This procedure offers effective treatment in whitening procedure but is often associated with teeth sensitivity, sore/injured gums, and bad taste of the product itself. U.S. Pat. No. 6,361,320 issued on Mar. 26, 2002 by Yarborough entitled “Method for Whitening Teeth” further elaborates on using high concentration of peroxides and light activation by laser with other pigments added to composition in order “to further increase of light energy absorbed by oxidizing molecules” Very often follow up treatment is recommended to prevent re-staining of the teeth after the bleaching. Take home bleaching kits are also recommended as a follow up treatment and have 8-10% concentration of hydrogen peroxide. Due to the painful side effects of bleaching, fluoride treatments are often prescribed to desensitize teeth see: “Bleaching for Various Complexions”, James M. Eisdorfer, DDS, Dentistry Today, Issue Date: July 2004”. Additional ingredients are often added to various bleaching compositions to decrease tooth sensitivity and/or sore gums following treatment.

“Relief”, a gel manufactured by Discus Dental Company contains potassium nitrate and fluoride is such a product, or amorphous calcium phosphate is added to bleaching formula: see their advertisement in “Dentistry Today” December 2004 page 146.Current invention offers an alternative, using lower concentration of hydrogen peroxide (H₂O₂). Typically concentration of H₂O₂ in candy is less than 2%, and can provide follow up and maintenance alternative. Use of candy will not require a dental appointment and will not aggravate the painful side effects of bleaching procedures.

Existing whitening products for home use are generally less effective than professional whitening because they use smaller concentration of hydrogen peroxide. These home products can go as low as 5.25% concentration of hydrogen peroxide, as seen in “Bright Smile” formula, and go as high as 22% like in Henry Schein's formula “New Natural Elegance” see: Dentistry today December 2004 page 120. Due to the lower concentrations of active ingredients, take home kits require several days' worth of application, and may have the side effects often associated with professional treatment. Treatments typically include bleaching trays, very often custom made in dental laboratories.

Some other systems such as “LumaLite” from Stay-Brite employ “paint on” technique that eliminates need for tray fabrication. These systems requires direct application of peroxide, usually carbamide (that is water soluble), two to three times a day for two weeks. “Paint on’ techniques are very often used as part of a maintenance system following a professional procedure. LumaLite can only be purchased from the dentist. “Dentistry Today” December 2004 Issue page 126. Typically they are applied on dry teeth surfaces and contain as an oxidizing agent carbamide. Brush on kits are not as effective as previous systems.

While the current invention relies on the chemical reaction between calcium peroxide (or any other peroxides) and citric acid in presences of saliva to generate low concentrations of the active ingredient, hydrogen peroxide. Additionally calcium from the reaction combined with salvia may play a positive role in re-mineralization of the surfaces of teeth that were leached out during professional treatment.

Whitening strips are intended as whitening product for use without a need for custom tray as a means to deliver whitening medium to the surfaces of the teeth. They are applied to dry surfaces of the teeth (as in paint products) and release whitening agent when hydrated. As with other systems peroxide is released to the surfaces of the teeth and is selected from groups containing hydrogen peroxide, carbamide peroxide, calcium peroxide, sodium percarbonate, sodium perborate, tetrasodium pyrophosphate peroxidate and combinations of thereof. See Pat. No 6,689,344 “Patches for Teeth Whitening” by Chang Sung Youn. Feb. 16, 2004 or wet method see U.S. Pat. No. 5,879,691 by Sagel Paul issued on Mar. 9, 1999 entitled: “Delivery-System for Tooth Whitener Using a Strip of Material Having Low-Flexural Stiffness” Typically strips are left in the mouth for several hours or overnight, and require several sessions to complete whitening procedure.

Although toothpastes contain bleaching agents, they rely mostly on the mechanical action of brushing for stain removal, and are augmented by polishing agents. Due to their short duration of contact with the surfaces of teeth (brushing time), toothpastes are the least effective form of tooth whitening products. see Pat. Nos. 4,603,045, 4,405,599, Toothpaste for Bonded (Composite Filling Material) as Well as Natural Teeth” by Smigel. Irwin. Jul. 29, 1986, 4,405,599, “Toothpaste for Natural Teeth as Well as Composite-Filling Material” Smigel Irwing Sep. 20, 1983. Smigel simply mentions titanium white as a whitening agent, without disclosing it as an active ingredient, or mechanism of its action. Most likely it is used to enhance the appearance of the product itself by making it look whiter. Calcium peroxide (calprox) is referred to as oxidizing agent on the patent itself together with sodium perborate. As a whitening agent calcium peroxide is mentioned only on the toothpaste itself, marketed under the brand name Supersmile. Brushing deposits toothpaste with bleaching and whitening agents over the surfaces of the teeth, while polishing agents: quarts, or barium glass removes stains mechanically. Additives augment the effectiveness of the toothpaste, while main objective is achieved by mechanically removing the stains. Smigel in Pat. No. 4,603,045 refers to Sodium bicarbonate as a cleaning and foaming agent, he also mentions it as an alkalizer without disclosing any connection with titanium white. Some other problems associated with whitening toothpaste are illustrated by U.S. Pat. No. 6,121,213 issued on Sep. 19, 2000 by Vergara Democria, entitled “Stable Peroxide Denture Paste” employs combination of bicarbonate and monoflourophospate to prevent degradation of peroxide during storage, other like U.S. Pat. No. 4,837,008 by Rudy Jerome issued on Jun. 6 1989 entitled “Periodontal composition and Methods” employs protective coating to protect peroxide from reaction during storage. “Whitening Mouth Wash” U.S. Pat. No 4,925,655, Smigel Irwing “Powder for Composition for-Forming a Mouthwash” May 15, 1990 is another product containing calcium peroxide as an active cleaning agent. The dry form in this composition is also another solution to prevent the deterioration of peroxides during storage by using anhydrous Calcium Peroxide and Sodium Bicarbonate.

U.S. Pat. No. 6,610,276 entitled “Multi-functional Dental composition” by Melman Steven issued Aug. 26, 2003, mentions organic acids in conjunction with abrasive powders as a means to achieve cleaning and brightening of the teeth.

My own patent U.S. Pat. No. 5,980,641 issued Nov. 9, 1999 and entitled “Methods and Solutions for Cleaning Dentures” establishes effectiveness of organic acids as active ingredients in tartar and stain removal agents, from dental appliances and its safety for the dental wearer and dental materials.

U.S. Pat. No. 6,471,945 issued on Oct. 29, 2002 by Luo Siuh and entitled “Stain Removing Chewing Gum and Confectionary Compositions, Methods of Making and Using Same” employs surfactants as an active cleaning agent. Present invention uses surfactants to facilitate better mixing of active ingredients to start chemical action. The reaction between calcium peroxide and citric acid will result in the release of hydrogen peroxide. The generation of hydrogen peroxide will assist in teeth bleaching.

Titanium white is mentioned as filler intended to improve attractiveness of the product itself. Current invention is using titanium white dissolved in alkaline solution (saliva) to coat the surfaces of the teeth and mask remaining stains.

“Edible Oral Cleansing” U.S. Pat. No. 5,843,471, Chaykin issued Dec. 1, 1998 mentions an lozenge, or candy comprising of as active ingredients: surfactants (glyceride or alkyl sulfate), sequestrants (citric acid) and precipitant especially Tannic acid (not a true acid), same ingredients are also mentioned in Chaykin's other Pat. No. 6,013,274, issued Jan. 11, 2000 entitled “Oral Cleansing: Methods and Compositions” and U.S. Pat. No. 6,090,404 issued Jul. 18, 2000 entitled “Oral Cleansing: Methods and Compositions” mentions citric acid as a means to stimulate saliva excretion.

Present invention utilizes citric acid to start a chemical reaction with calcium peroxide to produce hydrogen peroxide, in addition to enhanced saliva excretion. Calcium peroxide when in contact with saliva laden liquid citric acid, will release hydrogen peroxide, a bleaching agent.

Citric acid in preferred embodiment of present invention is included in a different part of the candy than the calcium peroxide (non-water soluble). It is sucking action in combination with saliva will dissolve the candy, and combines the two ingredients together, resulting in creating hydrogen peroxide. Bleaching action of the hydrogen peroxide is further enhanced by the alkaline center of the candy. Once the candy is dissolved, titanium dioxide (dissolved in alkaline solution) will coat the surfaces of teeth increasing their apparent whiteness. Optional usage of surfactants in present invention will not rely on surfactants as a source of cleaning. When used, surfactants will help to better mix and dissolve calcium peroxide with citric acids, and dissolved titanium white in aqueous sodium bicarbonate.

Wagenknecht Pat. No. 4,170,633 issued Oct. 9, 1979 and entitled “Plaque inhibiting composition and method” uses sodium bicarbonate as an alkaline buffer. While active cleaning ingredients are dental abrasives to mechanically remove stains while chewing gum

Also see his “Chewing Gum” Pat. No. 3,590,120, by Muhler Joseph Jun. 29 1971 Chewing gum containing whitening compounds such as calcium peroxide rely on mastication to release their bleaching agents, and combine them with citric acid, in order to release hydrogen peroxide see Pat. No. 6,696,043 “Teeth Whitening Composition in the Form of a Chewing Gum” by Orlowski Feb. 24, 2004. Present invention does not rely on mastication to combine and release the active ingredients together. Instead the present invention depends on saliva stimulation to release and combine active ingredients. Saliva allows for the release of non-water soluble calcium peroxide from one part of Isomalt based candy. Calcium peroxide is then combined with a mixture of saliva and citric acid (acidic solution) from another part of the candy thus releasing hydrogen peroxide as a bleaching and oxidizing agent.

Sucking on chewing gum will not dissolve the base of the gum, nor will it release its active components. Without chewing, whitening gum will not release hydrogen peroxide its active ingredient. Due to limited concentration of hydrogen peroxide in either product, there will be a necessity for repeated usage. There are several problems that use of gum may contribute to, and may contraindicate its usage. Chewing gum contributes to teeth loosening, bruxism, TMJ. In addition to contributing to medical conditions, chewing gum may not always be socially acceptable: easy disposal of used gum is not always available, possible perceived esthetical consequences of strengthening muscles of mastication in women, and finally chewing gum is not as discreet as sucking on candy. Furthermore chewing gum as a part of maintenance program following professional bleaching may exacerbate the sensitivity associated from the procedure itself. Sucking on candy tends to be less invasive and should not aggravate the painful side effects of professional cleaning. In addition the present invention uses titanium white to increase the apparent whiteness of teeth. Use of separated components in the candy is not intended as a means of extending the shelf life of the candy. The individual components are kept separate to prevent premature onset of chemical reaction.

Pat. No. 6,846,500 by Luo entitled “Oral Care Chewing Gums and Method of Use” issued on Jan. 25, 2005 mentions as a additional ingredient titanium dioxide, without mentioning and understanding the nature of dissolving. Titanium dioxide is normally water insoluble (see Van Nostrand Encyclopedia, New York 1983 page: 2837 line 22,) and will in turn coat and mask residual stains from the surfaces of the teeth.). Titanium white in Pat. No. 6,846,500 is again intended as a filler or as colorant for the product itself. Titanium white is simply being used to make the chewing gum more attractive in appearance to the consumer. Luo uses sodium bicarbonate as a cleaning agent, and not as means to dissolve titanium white.

Titanium dioxide of the present invention will dissolve in the alkaline portion of the candy. Luo only mentions the candy in contents of having specific moisture context. Moisture content is irrelevant to present invention because some of the active ingredients are non-soluble in water based solutions. They are imbedded separately and are only activated when the candy itself dissolves in the presence of saliva.

Additionally U.S. Pat. Nos. 6,485,739 and 6,696,044 by Luo entitled: “Stain Removing Chewing Gum and Confectionary Compositions and Methods of Making and Using same” relies upon surfactant to remove stains from the surfaces of the teeth. Present invention does not use surfactant as an active ingredient in whitening and stain removing candy. Surfactants are simply used for more efficient mixing of the active ingredients. U.S. Pat. No. 5,825,291 by Howard entitled “Chewing Gum Containing a Teeth Whitening Agent” issued on Oct. 20, 1998 use sodium bicarbonate as a cleaning and deodorizing agent. Citric acid as a hydrogen peroxide originator is not mentioned nor is it included as an ingredient in preferred embodiment. Instead the gum relies on I quote: “water soluble alkali metal percarbonate such as sodium or potassium percarbonate. . . . because it is highly water soluble and dissolves quickly to generate hydrogen peroxide and also provides source of alkalinity in the mouth” as active ingredient, while present formula uses inert and non-water soluble ingredients mixed together in presence of saliva to release bleaching agents. Insolubility in water of calcium peroxide and titanium white make the present formula more stable and moisture resistant.

Pat. No. 5,972,374 by Theisen entitled “Chewing Gum with Teeth Whitener” issued on Oct. 26, 1999 relies on whitening gel incorporated in a cavity of chewing gum, and not integral part of the composition, released by puncturing the cavity. Present invention does not rely on puncturing candy to release bleaching agent. Instead the present invention uses salivation to generate bleaching agent.

U.S. Pat. No. 6,416,744 by Robinson entitled: “Tooth Whitening Chewing Gum” relies on silica particles to polish off the stains. Titanium White is added as a colorant to make gum look more attractive for the buyer grouped together with flavoring agents and not necessarily as whitening agent. Present invention does not rely on mechanical removal of stains. Present invention does not use Titanium to improve the appearance of the product itself, titanium white is used for whitening of stained portions of teeth.

While toothpaste is effective in cleaning and to some degree whitens teeth, it needs additional aids: a toothbrush, source of water for rinsing (teeth and brush) and a place to dispose the used water. Usually tooth brushing is not performed in public thus further limiting its usage. Chewing gum is not universally socially acceptable and is unsightly for many people. Chewing gum is not risk free, it can contributes to loosening and premature tooth abrasion, bruxizm and contributes to TMJ (Temporomandibular Joint Syndrome). Gum has a tendency to stick especially to dental restorations and in addition provides problems with disposal of used up gum. One of side effects especially associated with home whitening systems is teeth sensitivity, and chewing gum exacerbates pain sensations and so on. Various acid components in teeth whitening products contribute to this problem by dissolving and eroding (leaching out) teeth structure and enamel, especially around the gingiva.

Teeth Whitening candy is free from these adverse side effects. Increased production of saliva is maintained even after the candy is fully consumed. The additional saliva helps in lowering the acidity of the mouth, washing out bacteria and food particles, hydrates the mouth, and decreases symptoms caused professional bleaching.

People with various diseases of the mouth (especially dry mouth syndrome) will be additional beneficiaries of the candy. Saliva excretion promoted by candy will improve positive feeling of general cleanness and mouth abnormalities associated with the diseases such as: ulcers, gingivitis, periodontal problems and not properly cleaned teeth due to tenderness will be gently moistened while beneficial effects of the candy will be introduced with saliva into the mouth.

Professional Cleaning is very costly, and requires both a dental appointment and follow up visit. It is often associated with tenderness of the teeth and gums, as well as sensitivity to heat and cold because of damage to teeth enamel. Cleaned teeth are more susceptible to secondary re-staining or rebound see: “Bleaching for Various Complexions”, James M. Eisdorfer, DDS, Dentistry Today, Issue Date: July 2004 and requires a maintenance program to protect clean and “white” teeth surfaces. And re-bleaching after 1-2 years. Professional bleaching causes sensitivity (Dentistry Today, Issue Date: July 2004, Posted On: Sep. 10, 2004:

### BRIEF SUMMARY OF INVENTION

The present invention relates to a new kind of teeth whitening product in a form of sugar free candy. Chemical composition of a candy will permit user a discreet and convenient method of whitening teeth with antibacterial, tartar and calculus removal properties. Present invention is intends as a stand alone product. Additional usages for the candy/lozenge include: maintenance product following professional cleaning and/or beaching of teeth, and breath freshening candy.

### DETAILED DESCRIPTION OF THE INVENTION

The present invention as stated above describes a sugar free product in a form of candy or lozenge, which will whiten teeth, inhibit plaque and tartar formation. Candy will freshen breath and coat the teeth surfaces with alkaline solution of titanium white, thus reinforcing apparent whiteness of the teeth. Above mentioned candy will comprise of active ingredients: organic acids (including citric acid), peroxides (including calcium peroxide), sodium bicarbonate, and titanium white. Inactive ingredients will include Isomalt, or any similar sugar free filler, sweeteners, flavorings including peppermint, or cinnamon flavor, surfactants. The exact proportions of the ingredients will be determined by taste and size of final product. Basic principal mode of action of the candy is as follows: Outer part of the candy made of Isomalt (or sugar free filler) will include and melt together two or more separate parts not mixed together. One separate part will consist of citric acid (or any combination of organic acids), flavorings, artificial sweeteners, surfactants (such as Sodium Laurel Phosphate). The other part in addition to above mentioned ingredients will include calcium peroxide (or any other peroxides). Sucking on candy will dissolve the filler (Isomalt), and allow for the release of calcium peroxide and citric acid. Once released, they will then undergo a chemical reaction and generate hydrogen peroxide, a bleaching agent and oxidant. Hydrogen peroxide will then bleach stains and oxidize proteins pellicles present on the surface of the teeth. While acidic saliva solution itself, will soften calcium deposits (tartar and calculus) on the surfaces of the teeth. Flavor and aromatic ingredients of the candy itself will promote saliva secretion and will help to wash out bacteria and dissolved calcium deposits from the teeth. With its antibacterial properties, hydrogen peroxide will oxidize odor causing bacteria. Additionally it will disrupt fermentation of food particles by bacteria responsible for tooth decay, thus improving overall dental hygiene.

In the preferred embodiment calcium peroxide concentrations will comprise of 0.5% to 5% by the weight of the candy. Citric acid concentrations will comprise from 1% to 5% by weight of the candy. The ingredients of the candy, both active and inactive, will be subject to adjustments dictated by taste and functionality of the candy itself.

The alkaline inner part of the candy will consist of titanium white (normally non-soluble in water) dissolved by aqueous solution of sodium bicarbonate (alkaline medium for dissolution). Included in the mixture will be flavoring agents and sweeteners. Additionally stimulation of the taste buds will occur by the addition of the citric acid to the solution resulting in formation of effervescence (C₂O₂ dissolved in water). Addition of organic gum (such as arabic or acacia gum) will thicken the solution and increases contact/reaction time with the surfaces of teeth. Titanium white is ordinarily non soluble in water, will dissolve in sodium bicarbonate. Once dissolved titanium white will coat and mask un-removed stains. It will also fill small fissures and depressions of teeth, increasing the apparent whiteness of teeth.

Addition of aromatic ingredients (such as peppermint or cinnamon oil) will freshen breath and will contribute to overall feeling of cleanness. All the ingredients will have to be carefully balanced in order to produce pleasant tasting candy. It will encourage its further use based on the taste of the candy itself in addition to its whitening action and benefits. Increased hydration of the mouth caused by sucking on candy will provide relief from dry mouth symptoms. Increased salivation will help with elimination of the bacteria responsible for tooth decay and bad odor. Flavorings such as Tannis (bitter tasting) could improve saliva excretions and will enhance mixing of dissolved components. Having separate components in the candy will allow distinct and diverse flavor combination (like mint and sour apple). Furthermore, it will decrease incidents of premature chemical activity due to outside conditions such as moisture, or heat.

### OTHER EMBODIMENT

Additional embodiment, the candy will be produced as a single unit by mixing all the ingredients together; resulting in a simplified production process. This will however limit taste combinations and possibly the efficacy of the product.

### OTHER EMBODIMENT

Additional embodiment, the candy will be produced as a single unit and used in conjunction with an ultrasonic appliance to clean removable dental appliance. The examples provided above are not meant to be exclusive. Many other variations of the present invention would be obvious to those skilled in the art, and are contemplated to be within the scope of the appended claims. For example changing concentrations of ingredients, adding/removing ingredients, adding/removing steps to manufacturing, and variations on the size of the candy will ultimately rely on the same basic principles found in the original embodiment. Consequently these variations will not change the principle (scope) of the invention.

What is claimed is:

1. Teeth whitening candy comprising of: Isomalt, citric acid (or any combination of organic acids), Tannic acid, calcium dioxide (or any other peroxides), sodium bicarbonate, titanium white, arabic gum (or any organic gum), sodium laurel phosphate (or any other surfactants), flavoring agents, sweeteners; capable of whitening the teeth, breath freshening and dissolving tartar and calculus when sucking

2. Teeth whitening candy of claim 1 will have separate components

3. Teeth whitening candy of claim 1 will have calcium dioxide as a separate ingredient in multiple part(s) of the candy

4. Teeth whitening candy of claim 1 will contain citric acid as separate ingredient in another part(s) of the candy

5. Teeth whitening candy of claim 1 will contain sodium bicarbonate and titanium white in the inner part(s) of the candy.

6. Teeth whitening candy of claim 5 will be center of the candy

7. Teeth whitening candy of claim 4 having citric acid or (any combination of organic acids) in concentration between 1% and 5% by weight of the candy

8. Teeth whitening candy of claim 4 having tannic acid in concentration between 0.5% and 1% by weight of the candy

9. Teeth whitening candy of claim 3 having calcium dioxide (or any other peroxides) in concentrations between 1% and 5% by weight of the candy.

10. Teeth whitening candy of claim 5 having sodium bicarbonate in concentrations 0.5% and 5% by weight of the candy

11. Teeth whitening candy of claim 5 having titanium white in concentrations 0.5% and 5% by weight of the candy

12. Teeth whitening candy of claim 1 having as a component a Sodium Laurel Phosphate, (or any other surfactants) will facilitate more efficient mixing of components.

13. Teeth whitening candy of claim 1 having citric acid (or combinations of organic acids), will stimulate the taste buds along the edges of the tongue thereby increasing salvation resulting in more effective mixing of organic acids and peroxides

14. Teeth whitening candy of claim 1 having flavoring added in a form of sweeteners will stimulate the taste buds along the front of the tongue thereby increasing salivation resulting in more effective mixing of organic acids and peroxides

15. Teeth whitening candy of claim 1 having tannic acid with its bitter taste will stimulate saliva excretions by acting upon taste buds in the back of the tongue resulting in more effective mixing of organic acids and peroxides

16. Teeth whitening candy of claim 1 having having added aromatic additives components (like sour apple aroma and menthol), or mint, will further stimulate excretion of saliva and will have breath freshening effect in candy.

17. Teeth whitening candy of claim 1, even after consumption, will still further stimulate excretion of saliva. Contributing to further flushing out of food debris, washing out bacteria, and aiding in the re-mineralization of teeth surfaces

18. Teeth whitening candy of claim 3 will release calcium dioxide upon dissolving

19. Teeth whitening candy of claim 4 will release citric acid upon dissolving

20. Teeth whitening candy of claim 4 having citric acid or any other organic acid will mix with calcium peroxide of claim 5 or any other peroxides resulting in creation of hydrogen peroxide

21. Teeth whitening candy of claim 20 will have antibacterial properties

22. Teeth whitening candy of claim 1 will have hydrogen peroxide as both a bleaching and oxidizing agent

23. Teeth whitening candy of claim 18 will have acidic saliva solution, contributing to dissolving calculus and tartar from the surfaces of the teeth

24. Teeth whitening candy of claim 1 containing sodium laurel phosphate will act as mild surfactant to further aid in mixing the organic acids and peroxides

25. Teeth whitening candy of claim 5 will dissolve sodium bicarbonate upon contact with saliva

26. Teeth whitening candy of claim 5 will release titanium white in contact with saliva

27. Teeth whitening candy of claim 5 will dissolve sodium bicarbonate in contact with saliva and will create alkaline solution

28. Teeth whitening candy of claim 5 will dissolve titanium white in contact with alkaline solution of sodium bicarbonate

29. Teeth whitening candy of claim 5 will mix dissolved titanium white with arabic gum (or any organic gum) to create thickened solution

30. Teeth whitening candy of claim 28 will cover teeth surfaces to mask un-dissolved stains

31. Teeth whitening candy of claim 28 will cover pits in teeth surfaces masking stains and brightening the teeth

32. Teeth whitening candy of claim 1 optionally could be created as a one unit with all ingredients already mixed together

33. Teeth whitening candy of claim 25 will mix with sodium bicarbonate, while dissolving and will combine in presence saliva with citric acid (or any organic acid) with bleaching agents will create C₂O₂ to further stimulate taste buds.

34. Teeth whitening candy of claim 5 with aromatic additives will act as a breath/mouth freshener

35. Teeth whitening candy of claim 1 will clean and dissolve tartar and calculus from The surfaces of dental appliances when used in conjunction with ultrasonic appliance
